(19) United States
(12) Patent Application Publication
(10) Pub. No.: US 2026/0290619 A1
(43) Pub. Date: Sep. 24, 2026

(54) SYSTEM AND METHOD FOR FACILITATING MEDICAL CHECKUPS AT VEHICLE CHARGING STATIONS

(71) Applicant: ShareSafe Solutions, LLC, Mobile, AL (US)

(72) Inventors: Robert B. Hanson, Mobile, AL (US); Dean Kei Naritoku, Mobile, AL (US); Peter Pronovost, Novelty, AL (US)

(73) Assignee: ShareSafe Solutions, LLC, Mobile, AL (US)

(21) Appl. No.: 19/474,683

(22) PCT Filed: Apr. 10, 2024
(86) PCT No.: PCT/US24/23950

§ 371 Date: Oct. 10, 2025

Related U.S. Application Data
(60) Provisional application No. 63/458,413, filed on Apr. 10, 2023.

(51) Int. Cl. G16H 80/00; G06F 9/451; G06T 7/00

(52) U.S. Cl. CPC G16H 80/00; G06T 7/0012; G06F 9/451; G06T 2200/24; G06T 2207/10016; G06T 2207/20081

(57) ABSTRACT

A system and method for facilitating virtual medical checkups is provided. Generally, a user may activate a display device of a medical charging/fueling station, which may then identify the user via a security means, such as logging into a user profile via a computing device operably connected to the display device, scanning a predefined pattern, and/or a secondary security device. A computer readable signal containing login credentials may be sent to the display device, allowing the user to access various applications thereon, including an application that may connect the user with healthcare personnel. In some embodiments, the system may allow a user to connect medical devices to the charging station to collect vitals of the user. In other embodiments, the system may provide additional security for electronic devices of a vehicle to reduce the likelihood of a security breach.

### CROSS REFERENCES

This application claims priority to U.S. Provisional Application Ser. No. 63/458,413, filed on Apr. 10, 2023, wherein said application is incorporated herein in its entirety by reference.

### FIELD OF THE DISCLOSURE

The subject matter of the present disclosure refers generally to a system and method for identifying users; securely logging into user applications of a vehicle and a computing device; and creating facilitating virtual medical checkups.

### BACKGROUND

The integration of healthcare services with routine activities presents an opportunity to enhance the accessibility and convenience of medical care. The modern lifestyle, characterized by a fast-paced environment and time-sensitive commitments, often leaves individuals with limited time to attend to their health and wellness. Regular visits to healthcare facilities for checkups and consultations are frequently postponed or neglected due to these time constraints. This neglect can lead to the exacerbation of health issues that could have been managed or mitigated with timely medical attention.

The advent of electric vehicles (EVs) has introduced a new dynamic into the daily routines of individuals. While EVs offer numerous benefits, including environmental friendliness and cost savings on fuel, they also require a considerable amount of time to charge, especially when compared to the time it takes to refuel a conventional gasoline vehicle. This charging time, often perceived as a period of inactivity or downtime, presents a window of opportunity that can be utilized for other productive tasks.

The concept of utilizing the charging time of electric vehicles for conducting virtual medical checkups is a novel approach that addresses the challenge of integrating health management into the busy schedules of individuals. By capitalizing on the time spent at charging stations, users can engage in virtual consultations with healthcare professionals, undergo routine checkups, and receive medical advice without the added burden of making separate appointments or traveling to medical facilities.

Virtual medical checkups facilitated at vehicle charging stations can leverage the advancements in telemedicine and digital health technologies. These technologies have already demonstrated their potential in providing remote healthcare services, improving patient engagement, and enabling continuous health monitoring. By integrating these technologies into the EV charging infrastructure, a seamless and efficient healthcare delivery model can be established, catering to the evolving demands of society.

Furthermore, the proposed system and method for facilitating medical checkups at vehicle charging stations can contribute to the early detection and management of health conditions. With the ability to conduct health assessments during vehicle charging sessions, individuals are more likely to engage in preventive healthcare practices, potentially reducing the incidence of chronic diseases and the overall burden on healthcare systems.

The highlighted text in the document underscores the pressing nature of the problem at hand. The scarcity of time for wellness visits, juxtaposed with the substantial charging duration of electric vehicles, underscores the pressing demand for an innovative solution that can merge these two aspects of modern life. The proposed system and method aim to fill this gap by providing a dual-benefit model that enhances the utility of time spent at EV charging stations, transforming it into an opportunity for health maintenance and medical consultation.

In summary, the background of the present disclosure sets the stage for a transformative system and method that aligns with the contemporary trends of healthcare and automotive industries. It proposes a solution that not merely acknowledges the constraints of modern living but also offers a practical and innovative approach to overcome them. By doing so, it paves the way for a future where health management is integrated into the fabric of daily life, ensuring that wellness checks are no longer sidelined but are instead conveniently woven into the routine activities of individuals.

### SUMMARY

The present disclosure provides a system and method for facilitating virtual medical checkups at vehicle charging stations, enabling users to efficiently utilize the charging time of their electric vehicles (EVs) to engage in health maintenance activities. This innovative approach integrates telemedicine and digital health technologies with the EV charging infrastructure, offering a dual-benefit model that enhances the utility of time spent during vehicle charging sessions.

The system comprises a processor operably connected to at least one display, which is part of a medical charging/fueling station. A computing device with a user interface allows a user to initiate a virtual medical checkup on the display. Additionally, a medical device connected to the processor collects patient data from the user. The system also includes a non-transitory computer-readable medium coupled to the processor, containing instructions that, when executed, facilitate the virtual medical checkup process.

Operations performed by the processor include receiving a medical checkup request from the computing device, determining the appropriate display for presenting a healthcare professional feed, receiving patient data from the medical device, transferring the patient data to the healthcare professional, and presenting the healthcare professional feed on the display. This enables real-time interaction between the user and healthcare professional during the EV charging process.

The system further provides a means for securely identifying users and logging into user applications of a vehicle and a computing device. Security measures may include user profile login, scanning predefined patterns, and secondary security devices to ensure authorized access to the system and applications.

By leveraging the charging duration of EVs, the system encourages users to participate in preventive healthcare practices, potentially reducing the incidence of chronic diseases and the overall burden on healthcare systems. The system and method align with contemporary trends in healthcare and automotive industries, offering a practical solution to the constraints of modern living and integrating health management into the fabric of daily life.

The foregoing summary has outlined some features of the system and method of the present disclosure so that those skilled in the pertinent art may better understand the detailed description that follows. Additional features that form the subject of the claims will be described hereinafter. Those skilled in the pertinent art should appreciate that they can readily utilize these features for designing or modifying other structures for carrying out the same purpose of the system and method disclosed herein. Those skilled in the pertinent art should also realize that such equivalent designs or modifications do not depart from the scope of the system and method of the present disclosure.

### DESCRIPTION OF THE DRAWINGS

These and other features, aspects, and advantages of the present disclosure will become better understood with regard to the following description, appended claims, and accompanying drawings where:

FIG. 1 illustrates a system embodying features consistent with the principles of the present disclosure.

FIG. 2 illustrates a system embodying features consistent with the principles of the present disclosure.

FIG. 3 illustrates a system embodying features consistent with the principles of the present disclosure.

FIG. 4 illustrates a system for facilitating virtual medical checkups consistent with the principles of the present disclosure.

FIG. 5 illustrates display user interfaces of a system for facilitating virtual medical checkups and embodying features consistent with the principles of the present disclosure.

FIG. 6A illustrates a charging station configured for facilitating virtual medical checkups and embodying features consistent with the principles of the present disclosure.

FIG. 6B illustrates a charging station configured for facilitating virtual medical checkups and embodying features consistent with the principles of the present disclosure.

FIG. 6C illustrates a vehicle display configured for communicating with a charging station to facilitate virtual medical checkups and embodying features consistent with the principles of the present disclosure.

FIG. 7A illustrates an energy station comprising a plurality of charging stations and embodying features consistent with the principles of the present disclosure.

FIG. 7B illustrates a charging station comprising a privacy room and embodying features consistent with the principles of the present disclosure.

FIG. 8 illustrates the manner in which individual access to data may be granted or limited based on user roles and administrator roles.

### DETAILED DESCRIPTION

In the Summary above and in this Detailed Description, and the claims below, and in the accompanying drawings, reference is made to particular features, including method steps, of the invention. It is to be understood that the disclosure of the invention in this specification includes all possible combinations of such particular features. For instance, where a particular feature is disclosed in the context of a particular aspect or embodiment of the invention, or a particular claim, that feature can also be used, to the extent possible, in combination with/or in the context of other particular aspects of the embodiments of the invention, and in the invention generally.

The term “comprises”, and grammatical equivalents thereof are used herein to mean that other components, steps, etc. are optionally present. For instance, a system “comprising” components A, B, and C can contain only components A, B, and C, or can contain not only components A, B, and C, but also one or more other components. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility). As will be evident from the disclosure provided below, the present invention satisfies the need for a system and method capable of allowing users to undergo medical examinations at medical charging/fueling stations.

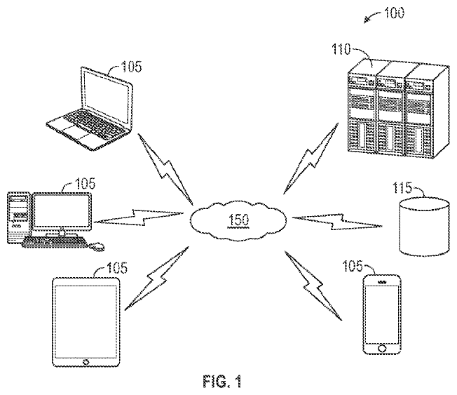

FIG. 1 depicts an exemplary environment 100 of the system 400 consisting of clients 105 connected to a server 110 and/or database 115 via a network 150. Clients 105 are devices of users 405 that may be used to access servers 110 and/or databases 115 through a network 150. A network 150 may comprise of one or more networks of any kind, including, but not limited to, a local area network (LAN), a wide area network (WAN), metropolitan area networks (MAN), a telephone network, such as the Public Switched Telephone Network (PSTN), an intranet, the Internet, a memory device, another type of network, or a combination of networks. In a preferred embodiment, computing entities 200 may act as clients 105 for a user 405. For instance, a client 105 may include a personal computer, a wireless telephone, a streaming device, a “smart” television, a personal digital assistant (PDA), a laptop, a smart phone, a tablet computer, or another type of computation or communication interface 280. Servers 110 may include devices that access, fetch, aggregate, process, search, provide, and/or maintain documents. Although FIG. 1 depicts a preferred embodiment of an environment 100 for the system 400, in other implementations, the environment 100 may contain fewer components, different components, differently arranged components, and/or additional components than those depicted in FIG. 1. Alternatively, or additionally, one or more components of the environment 100 may perform one or more other tasks described as being performed by one or more other components of the environment 100.

As depicted in FIG. 1, one embodiment of the system 400 may comprise a server 110. Although shown as a single server 110 in FIG. 1, a server 110 may, in some implementations, be implemented as multiple devices interlinked together via the network 150, wherein the devices may be distributed over a large geographic area and performing different functions or similar functions. For instance, two or more servers 110 may be implemented to work as a single server 110 performing the same tasks. Alternatively, one server 110 may perform the functions of multiple servers 110. For instance, a single server 110 may perform the tasks of a web server and an indexing server 110. Additionally, it is understood that multiple servers 110 may be used to operably connect the processor 220 to the database 115 and/or other content repositories. The processor 220 may be operably connected to the server 110 via wired or wireless connection. Types of servers 110 that may be used by the system 400 include, but are not limited to, search servers, document indexing servers, and web servers, or any combination thereof.

Search servers may include one or more computing entities 200 designed to implement a search engine, such as a documents/records search engine, general webpage search engine, etc. Search servers may, for instance, include one or more web servers designed to receive search queries and/or inputs from users 405, search one or more databases 115 in response to the search queries and/or inputs, and provide documents or information, relevant to the search queries and/or inputs, to users 405. In some implementations, search servers may include a web search server that may provide webpages to users 405, wherein a provided webpage may include a reference to a web server at which the desired information and/or links are located. The references to the web server at which the desired information is located may be included in a frame and/or text box, or as a link to the desired information/document. Document indexing servers may include one or more devices designed to index documents available through networks 150. Document indexing servers may access other servers 110, such as web servers that host content, to index the content. In some implementations, document indexing servers may index documents/records stored by other servers 110 connected to the network 150. Document indexing servers may, for instance, store and index content, information, and documents relating to user accounts and user-generated content. Web servers may include servers 110 that provide webpages to clients 105. For instance, the webpages may be HTML-based webpages. A web server may host one or more websites. As used herein, a website may refer to a collection of related webpages. Frequently, a website may be associated with a single domain name, although some websites may potentially encompass more than one domain name. The concepts described herein may be applied on a per-website basis. Alternatively, in some implementations, the concepts described herein may be applied on a per-webpage basis.

As used herein, a database 115 refers to a set of related data and the way it is organized. Access to this data is usually provided by a database management system (DBMS) consisting of an integrated set of computer software that allows users 405 to interact with one or more databases 115 and provides access to all of the data contained in the database 115. The DBMS provides various functions that allow entry, storage and retrieval of large quantities of information and provides ways to manage how that information is organized. Because of the close relationship between the database 115 and the DBMS, as used herein, the term database 115 refers to both a database 115 and DBMS.

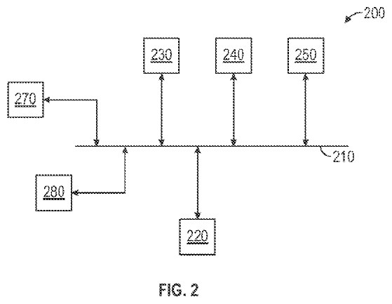

FIG. 2 is an exemplary diagram of a client 105, server 110, and/or or database 115 (hereinafter collectively referred to as “computing entity 200”), which may correspond to one or more of the clients 105, servers 110, and databases 115 according to an implementation consistent with the principles of the invention as described herein. The computing entity 200 may comprise a bus 210, a processor 220, memory 304, a storage device 250, a peripheral device 270, and a communication interface 280 (such as wired or wireless communication device). The bus 210 may be defined as one or more conductors that permit communication among the components of the computing entity 200. The processor 220 may be defined as logic circuitry that responds to and processes the basic instructions that drive the computing entity 200. Memory 304 may be defined as the integrated circuitry that stores information for immediate use in a computing entity 200. A peripheral device 270 may be defined as any hardware used by a user 405 and/or the computing entity 200 to facilitate communicate between the two. A storage device 250 may be defined as a device used to provide mass storage to a computing entity 200. A communication interface 280 may be defined as any transceiver-like device that enables the computing entity 200 to communicate with other devices and/or computing entities 200.

The bus 210 may comprise a high-speed interface 308 and/or a low-speed interface 312 that connects the various components together in a way such they may communicate with one another. A high-speed interface 308 manages bandwidth-intensive operations for computing device 300, while a low-speed interface 312 manages lower bandwidth-intensive operations. In some preferred embodiments, the high-speed interface 308 of a bus 210 may be coupled to the memory 304, display 316, and to high-speed expansion ports 310, which may accept various expansion cards such as a graphics processing unit (GPU). In other preferred embodiments, the low-speed interface 312 of a bus 210 may be coupled to a storage device 250 and low-speed expansion ports 314. The low-speed expansion ports 314 may include various communication ports, such as USB, Bluetooth, Ethernet, wireless Ethernet, etc. Additionally, the low-speed expansion ports 314 may be coupled to one or more peripheral devices 270, such as a keyboard, pointing device, scanner, and/or a networking device, wherein the low-speed expansion ports 314 facilitate the transfer of input data from the peripheral devices 270 to the processor 220 via the low-speed interface 312.

The processor 220 may comprise any type of conventional processor or microprocessor that interprets and executes computer readable instructions. The processor 220 is configured to perform the operations disclosed herein based on instructions stored within the system 400. The processor 220 may process instructions for execution within the computing entity 200, including instructions stored in memory 304 or on a storage device 250, to display graphical information for a graphical user interface (GUI) on an external peripheral device 270, such as a display 316. The processor 220 may provide for coordination of the other components of a computing entity 200, such as control of user interfaces 411, 511, 711, applications run by a computing entity 200, and wireless communication by a communication interface 280 of the computing entity 200. The processor 220 may be any processor or microprocessor suitable for executing instructions. In some embodiments, the processor 220 may have a memory device therein or coupled thereto suitable for storing the data, content, or other information or material disclosed herein. In some instances, the processor 220 may be a component of a larger computing entity 200. A computing entity 200 that may house the processor 220 therein may include, but are not limited to, laptops, desktops, workstations, personal digital assistants, servers 110, mainframes, cellular telephones, tablet computers, smart televisions, streaming devices, or any other similar device. Accordingly, the inventive subject matter disclosed herein, in full or in part, may be implemented or utilized in devices including, but are not limited to, laptops, desktops, workstations, personal digital assistants, servers 110, mainframes, cellular telephones, tablet computers, smart televisions, streaming devices, or any other similar device.

Memory 304 stores information within the computing device 300. In some preferred embodiments, memory 304 may include one or more volatile memory units. In another preferred embodiment, memory 304 may include one or more non-volatile memory units. Memory 304 may also include another form of computer-readable medium, such as a magnetic, solid state, or optical disk. For instance, a portion of a magnetic hard drive may be partitioned as a dynamic scratch space to allow for temporary storage of information that may be used by the processor 220 when faster types of memory, such as random-access memory (RAM), are in high demand. A computer-readable medium may refer to a non-transitory computer-readable memory device. A memory device may refer to storage space within a single storage device 250 or spread across multiple storage devices 250. The memory 304 may comprise main memory 230 and/or read only memory (ROM) 240. In a preferred embodiment, the main memory 230 may comprise RAM or another type of dynamic storage device 250 that stores information and instructions for execution by the processor 220. ROM 240 may comprise a conventional ROM device or another type of static storage device 250 that stores static information and instructions for use by processor 220. The storage device 250 may comprise a magnetic and/or optical recording medium and its corresponding drive.

As mentioned earlier, a peripheral device 270 is a device that facilitates communication between a user 405 and the processor 220. The peripheral device 270 may include, but is not limited to, an input device and/or an output device. As used herein, an input device may be defined as a device that allows a user 405 to input data and instructions that is then converted into a pattern of electrical signals in binary code that are comprehensible to a computing entity 200. An input device of the peripheral device 270 may include one or more conventional devices that permit a user 405 to input information into the computing entity 200, such as a controller, scanner, phone, camera, scanning device, keyboard, a mouse, a pen, voice recognition and/or biometric mechanisms, etc. As used herein, an output device may be defined as a device that translates the electronic signals received from a computing entity 200 into a form intelligible to the user 405. An output device of the peripheral device 270 may include one or more conventional devices that output information to a user 405, including a display 316, a printer, a speaker, an alarm, a projector, etc. Additionally, storage devices 250, such as CD-ROM drives, and other computing entities 200 may act as a peripheral device 270 that may act independently from the operably connected computing entity 200. For instance, a streaming device may transfer data to a smartphone, wherein the smartphone may use that data in a manner separate from the streaming device.

The storage device 250 is capable of providing the computing entity 200 mass storage. In some embodiments, the storage device 250 may comprise a computer-readable medium such as the memory 304, storage device 250, or memory 304 on the processor 220. A computer-readable medium may be defined as one or more physical or logical memory devices and/or carrier waves. Devices that may act as a computer readable medium include, but are not limited to, a hard disk device, optical disk device, tape device, flash memory or other similar solid-state memory device, or an array of devices, including devices in a storage area network or other configurations. Examples of computer-readable mediums include, but are not limited to, magnetic media such as hard disks, floppy disks, and magnetic tape; optical media such as CD ROM discs and DVDs; magneto-optical media such as optical discs; and hardware devices that are specially configured to store and perform programming instructions, such as ROM 240, RAM, flash memory, and the like.

In an embodiment, a computer program may be tangibly embodied in the storage device 250. The computer program may contain instructions that, when executed by the processor 220, performs one or more steps that comprise a method, such as those methods described herein. The instructions within a computer program may be carried to the processor 220 via the bus 210. Alternatively, the computer program may be carried to a computer-readable medium, wherein the information may then be accessed from the computer-readable medium by the processor 220 via the bus 210 as needed. In a preferred embodiment, the software instructions may be read into memory 304 from another computer-readable medium, such as data storage device 250, or from another device via the communication interface 280. Alternatively, hardwired circuitry may be used in place of or in combination with software instructions to implement processes consistent with the principles as described herein. Thus, implementations consistent with the invention as described herein are not limited to any specific combination of hardware circuitry and software.

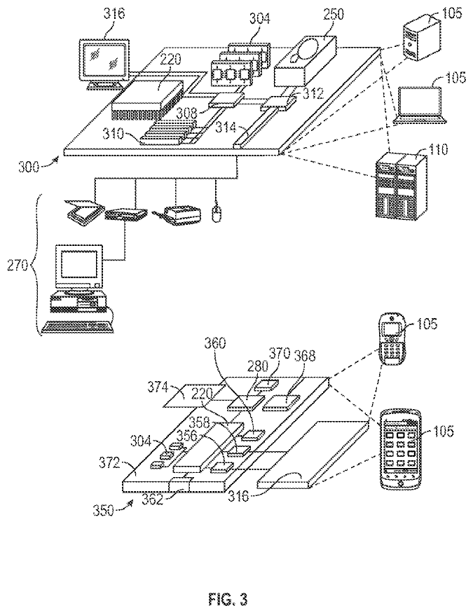

FIG. 3 depicts exemplary computing entities 200 in the form of a computing device 300 and mobile computing device 350, which may be used to carry out the various embodiments of the invention as described herein. A computing device 300 is intended to represent various forms of digital computers, such as laptops, desktops, workstations, servers 110, databases 115, mainframes, and other appropriate computers. A mobile computing device 350 is intended to represent various forms of mobile devices, such as scanners, scanning devices, personal digital assistants, cellular telephones, smart phones, tablet computers, and other similar devices. The various components depicted in FIG. 3, as well as their connections, relationships, and functions are meant to be examples only, and are not meant to limit the implementations of the invention as described herein. The computing device 300 may be implemented in a number of different forms, as shown in FIGS. 1 and 3. For instance, a computing device 300 may be implemented as a server 110 or in a group of servers 110. Computing devices 300 may also be implemented as part of a rack server system. In addition, a computing device 300 may be implemented as a personal computer, such as a desktop computer or laptop computer. Alternatively, components from a computing device 300 may be combined with other components in a mobile device, thus creating a mobile computing device 350. Each mobile computing device 350 may contain one or more computing devices 300 and mobile devices, and an entire system may be made up of multiple computing devices 300 and mobile devices communicating with each other as depicted by the mobile computing device 350 in FIG. 3. The computing entities 200 consistent with the principles of the invention as disclosed herein may perform certain receiving, communicating, generating, output providing, correlating, and storing operations as needed to perform the various methods as described in greater detail below.

In the embodiment depicted in FIG. 3, a computing device 300 may include a processor 220, memory 304 a storage device 250, high-speed expansion ports 310, low-speed expansion ports 314, and bus 210 operably connecting the processor 220, memory 304, storage device 250, high-speed expansion ports 310, and low-speed expansion ports 314. In one preferred embodiment, the bus 210 may comprise a high-speed interface 308 connecting the processor 220 to the memory 304 and high-speed expansion ports 310 as well as a low-speed interface 312 connecting to the low-speed expansion ports 314 and the storage device 250. Because each of the components are interconnected using the bus 210, they may be mounted on a common motherboard as depicted in FIG. 3 or in other manners as appropriate. The processor 220 may process instructions for execution within the computing device 300, including instructions stored in memory 304 or on the storage device 250. Processing these instructions may cause the computing device 300 to display graphical information for a GUI on an output device, such as a display 316 coupled to the high-speed interface 308. In other implementations, multiple processors and/or multiple buses may be used, as appropriate, along with multiple memory units and/or multiple types of memory. Additionally, multiple computing devices may be connected, wherein each device provides portions of the necessary operations.

A mobile computing device 350 may include a processor 220, memory 304 a peripheral device 270 (such as a display 316, a communication interface 280, and a transceiver 368, among other components). A mobile computing device 350 may also be provided with a storage device 250, such as a micro-drive or other previously mentioned storage device 250, to provide additional storage. Preferably, each of the components of the mobile computing device 350 are interconnected using a bus 210, which may allow several of the components of the mobile computing device 350 to be mounted on a common motherboard as depicted in FIG. 3 or in other manners as appropriate. In some implementations, a computer program may be tangibly embodied in an information carrier. The computer program may contain instructions that, when executed by the processor 220, perform one or more methods, such as those described herein. The information carrier is preferably a computer-readable medium, such as memory, expansion memory 374, or memory 304 on the processor 220 such as ROM 240, that may be received via the transceiver or external interface 362. The mobile computing device 350 may be implemented in a number of different forms, as shown in FIG. 3. For instance, a mobile computing device 350 may be implemented as a cellular telephone, part of a smart phone, personal digital assistant, or other similar mobile device.

The processor 220 may execute instructions within the mobile computing device 350, including instructions stored in the memory 304 and/or storage device 250. The processor 220 may be implemented as a chipset of chips that may include separate and multiple analog and/or digital processors. The processor 220 may provide for coordination of the other components of the mobile computing device 350, such as control of the user interfaces 411, 511, 711, applications run by the mobile computing device 350, and wireless communication by the mobile computing device 350. The processor 220 of the mobile computing device 350 may communicate with a user 405 through the control interface 358 coupled to a peripheral device 270 and the display interface 356 coupled to a display 316. The display 316 of the mobile computing device 350 may include, but is not limited to, Liquid Crystal Display (LCD), Light Emitting Diode (LED) display, Organic Light Emitting Diode (OLED) display, and Plasma Display Panel (PDP), holographic displays, augmented reality displays, virtual reality displays, or any combination thereof. The display interface 356 may include appropriate circuitry for causing the display 316 to present graphical and other information to a user 405. The control interface 358 may receive commands from a user 405 via a peripheral device 270 and convert the commands into a computer readable signal for the processor 220. In addition, an external interface 362 may be provided in communication with processor 220, which may enable near area communication of the mobile computing device 350 with other devices. The external interface 362 may provide for wired communications in some implementations or wireless communication in other implementations. In a preferred embodiment, multiple interfaces may be used in a single mobile computing device 350 as is depicted in FIG. 3.

Memory 304 stores information within the mobile computing device 350. Devices that may act as memory 304 for the mobile computing device 350 include, but are not limited to computer-readable media, volatile memory, and non-volatile memory. Expansion memory 374 may also be provided and connected to the mobile computing device 350 through an expansion interface 372, which may include a Single In-Line Memory Module (SIM) card interface or micro secure digital (Micro-SD) card interface. Expansion memory 374 may include, but is not limited to, various types of flash memory and non-volatile random-access memory (NVRAM). Such expansion memory 374 may provide extra storage space for the mobile computing device 350. In addition, expansion memory 374 may store computer programs or other information that may be used by the mobile computing device 350. For instance, expansion memory 374 may have instructions stored thereon that, when carried out by the processor 220, cause the mobile computing device 350 perform the methods described herein. Further, expansion memory 374 may have secure information stored thereon; therefore, expansion memory 374 may be provided as a security module for a mobile computing device 350, wherein the security module may be programmed with instructions that permit secure use of a mobile computing device 350. In addition, expansion memory 374 having secure applications and secure information stored thereon may allow a user 405 to place identifying information on the expansion memory 374 via the mobile computing device 350 in a non-hackable manner.

A mobile computing device 350 may communicate wirelessly through the communication interface 280, which may include digital signal processing circuitry where necessary. The communication interface 280 may provide for communications under various modes or protocols, including, but not limited to, Global System Mobile Communication (GSM), Short Message Services (SMS), Enterprise Messaging System (EMS), Multimedia Messaging Service (MMS), Code Division Multiple Access (CDMA), Time Division Multiple Access (TDMA), Personal Digital Cellular (PDC), Wideband Code Division Multiple Access (WCDMA), IMT Multi-Carrier (CDMAX 0), and General Packet Radio Service (GPRS), or any combination thereof. Such communication may occur, for example, through a transceiver 368. Short-range communication may occur, such as using a Bluetooth, WIFI, or other such transceiver 368. In addition, a Global Positioning System (GPS) receiver module 370 may provide additional navigation-and location-related wireless data to the mobile computing device 350, which may be used as appropriate by applications running on the mobile computing device 350. Alternatively, the mobile computing device 350 may communicate audibly using an audio codec 360, which may receive spoken information from a user 405 and covert the received spoken information into a digital form that may be processed by the processor 220. The audio codec 360 may likewise generate audible sound for a user 405, such as through a speaker, e.g., in a handset of mobile computing device 350. Such sound may include sound from voice telephone calls, recorded sound such as voice messages, music files, etc. Sound may also include sound generated by applications operating on the mobile computing device 350.

The system 400 may comprise a power supply, which may be any source of power that provides the system 400 with the required energy. In a preferred embodiment, the power supply may be a stationary power source that has been installed in a way such that it is fastened in place, such as a 3-prong wall outlet. In a preferred embodiment, the stationary power source is connected to the wiring system of a premises, such as a house or a building. In another preferred embodiment, the power supply may be a mobile power source, such as a battery pack, gas-powered generator, alternator, and fuel cell. In a preferred embodiment, the mobile power source does not need to be connected to the wiring system of a premises to provide power to the system 400 but may be capable of connecting to the wiring system of said premises to provide power to a system 400 connected thereto. In another preferred embodiment, the system 400 may comprise multiple power supplies configured to supply power to the system 400 in different circumstances. For instance, the system 400 may be directly plugged into a stationary power source, which may provide power to the system 400 so long as the system 400 does not move out of range of said stationary power source, as well as connected to a mobile power source, which may provide power to the system 400 when the system 400 is not connected to a stationary power source or in situations where the stationary power source ceases to provide power to the system 400. In yet another preferred embodiment, a plurality of solar charging panels may be operably connected to a battery of the system 400, which may then supply power to the system 400 either directly or via the wiring of the premises. As such, the system 400 may be configured to receive power in a variety of ways without departing from the inventive subject matter described herein.

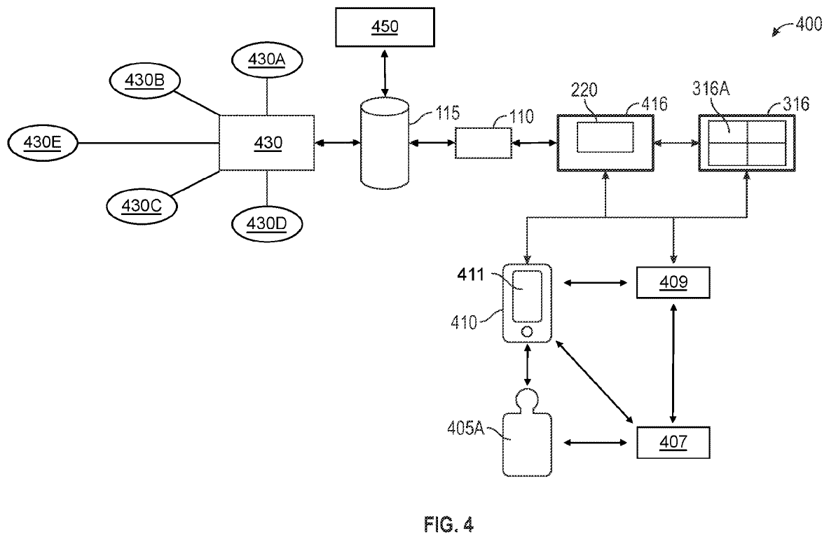

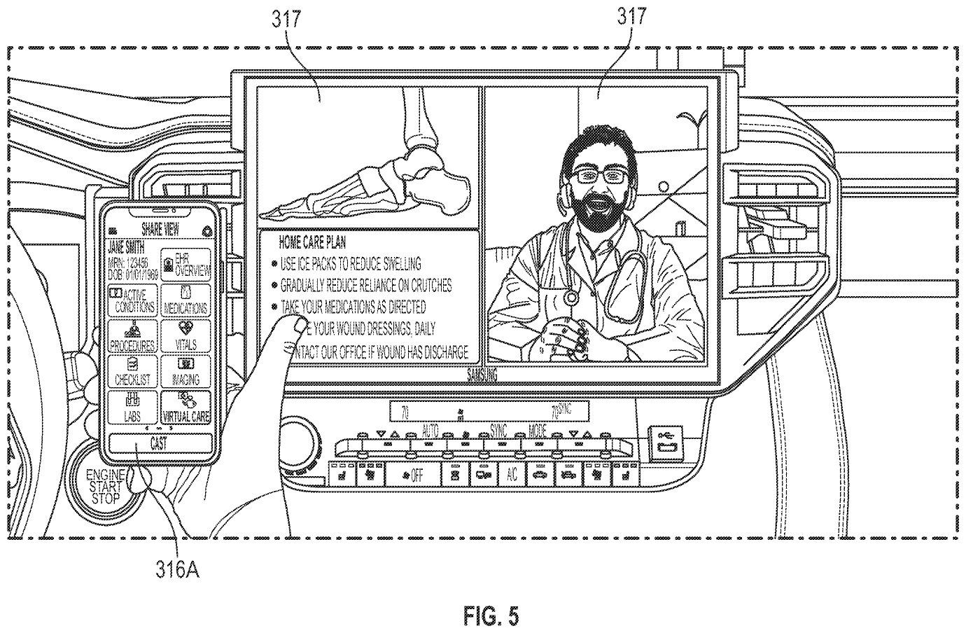

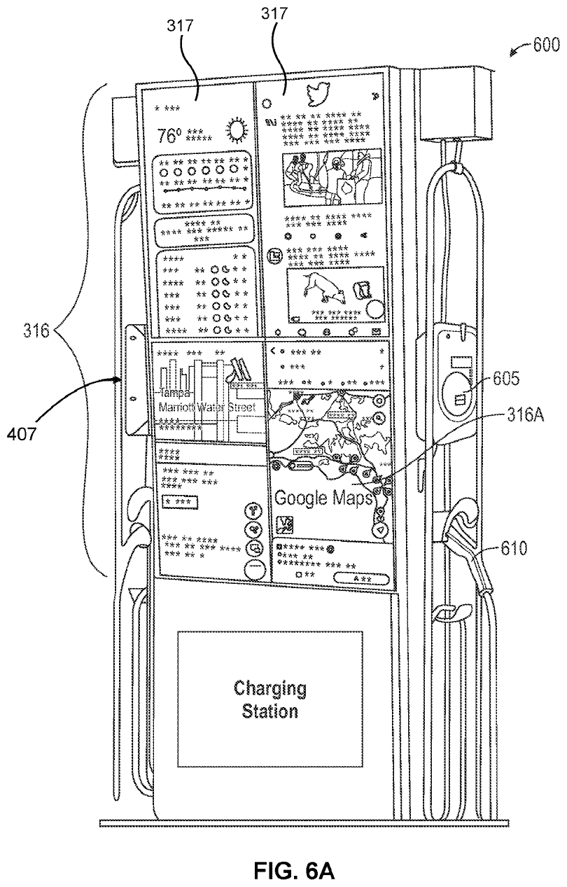

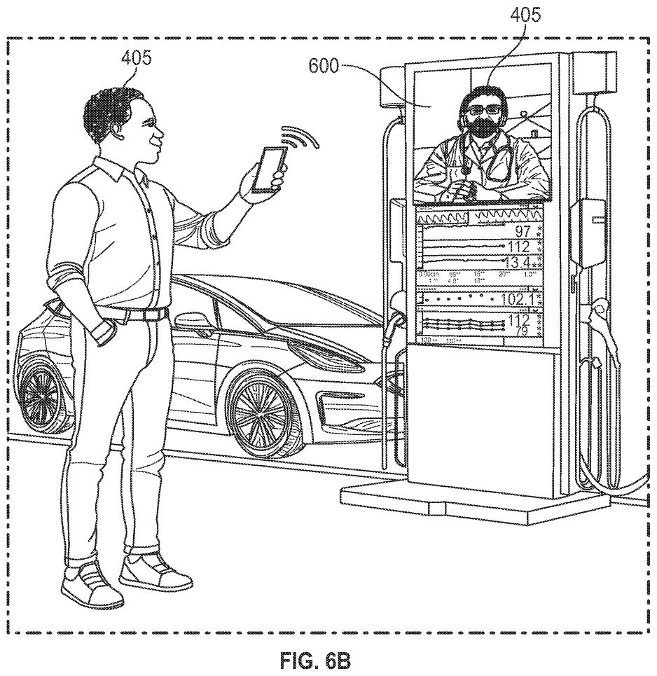

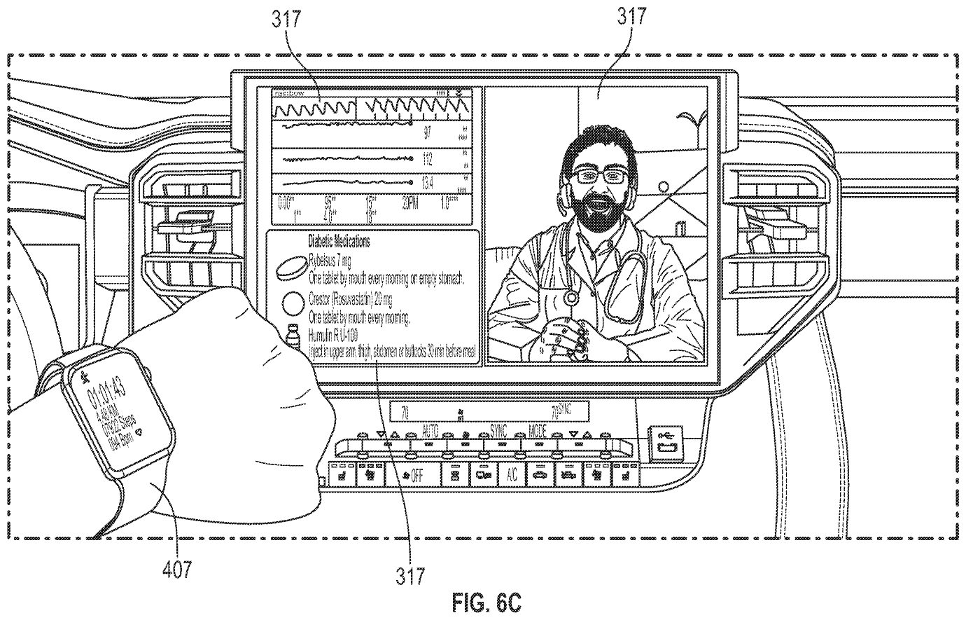

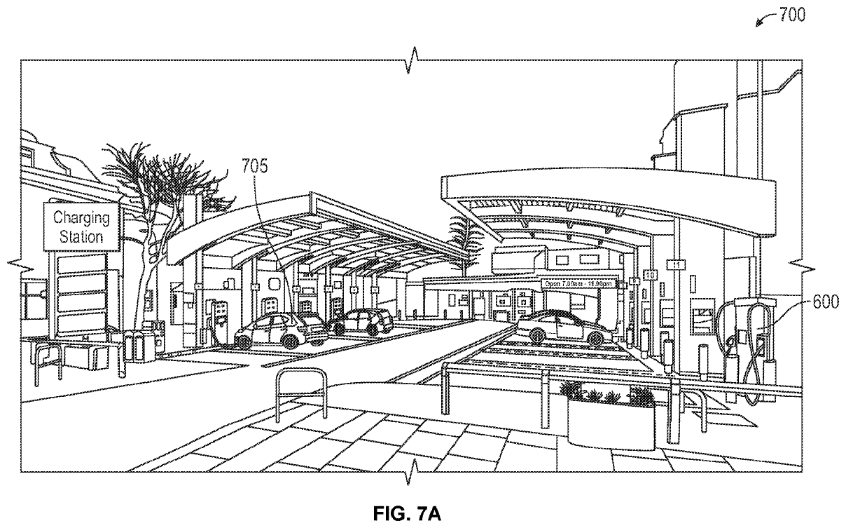

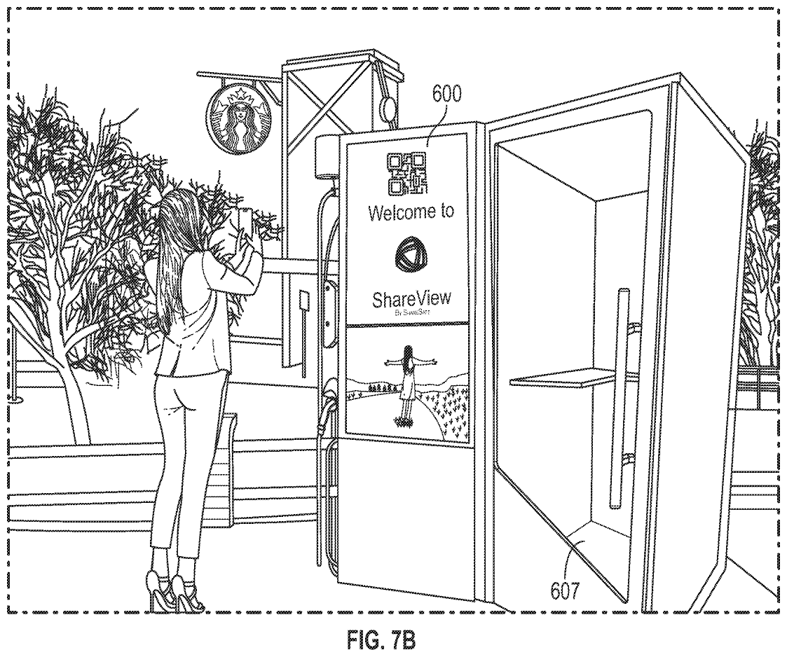

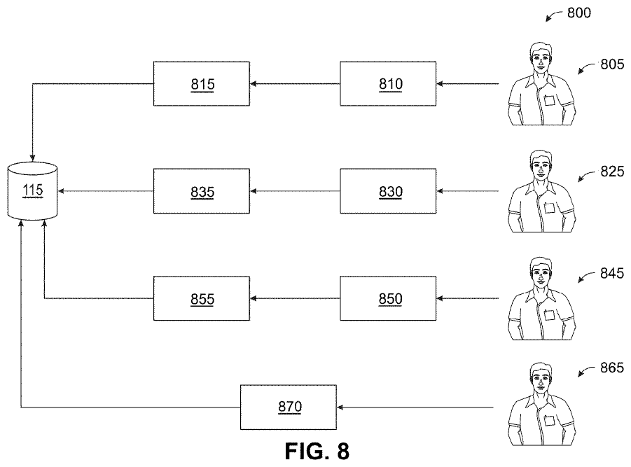

FIGS. 4-8 illustrate embodiments of a system 400 and methods for identifying a user 405; securely logging into user applications based on the identity of said user 405; and controlling a display 316 and/or electronics systems of a vehicle of said user 405, including facilitating the transfer of information between a user 405 and healthcare provider during a virtual medical checkup. FIG. 4 illustrates a preferred embodiment of the system 400 having a computing entity 200, display 316, and medical charging/fueling station 600 in fluid communication with one another. FIG. 5 illustrates an example user interface 411 of the computing entity 200, wherein a display 316 of a vehicle operably connected to a display of the medical charging/fueling station and/or computing entity of the user may split a display user interface 316A of the display of the vehicle into multiple display windows 317 containing applications of the display 316, applications of medical charging/fueling station 600, and/or applications of the computing entity 200 of the user 405. FIG. 6A-6C illustrate an example display user interface 316A of a display 316 of the medical charging/fueling station 600 or vehicle in direct communication with said medical charging/fueling station 600, wherein a control board 409 operably connected to said displays 316 facilitates the transfer of information therebetween. FIG. 7A illustrates the system 400 being used by a user 405 within an environment 700 to charge an electric vehicle 705 while receiving a virtual medical checkup. FIG. 7B illustrates a medical charging/fueling station further comprising a privacy room 607, providing users 405 of the system 400 with more privacy. FIG. 8 illustrates permission levels 800 that may be utilized by the present system 400 for controlling access to user content 815, 835, 855 such as user data 430A, image data 430B, application data 430C, patient data 430D, and display data 430E. It is understood that the various method steps associated with any methods of the present disclosure may be carried out as operations by the system 400 shown in FIGS. 4-8.

The system 400 generally comprises a computing entity 200 having a user interface 411, medical device 407 having at least one sensor configured to measure patient data 430D of a user 405, display 316 operably connected to said computing entity 200 and said medical device 407 and integrated into a medical charging/fueling station 600, processor 220 operably connected to said computing entity 200, medical device 407, and display 316, and non-transitory computer-readable medium (CRM) 416 coupled to said processor 220 and having instructions stored thereon. Some preferred embodiments may further comprise a camera 905 operably connected to computing entities 200, displays 316, and/or secondary security devices. In one preferred embodiment, a database 115 may be operably connected to the processor 220 and the various data of the system 400 may be stored therein, including, but not limited to, user data 430A, image data 430B, application data 430C, patient data 430D, and display data 430E. In some preferred embodiments, the displays 316 may further comprise a display user interface 316A having a plurality of display windows 317 configured to present the various data of the system 400 therein, wherein control boards 409 of the displays 316 may be configured to receive the various data of the system 400 and arrange the plurality of display windows 317 within the display user interface 316A. In yet another preferred embodiment, a wireless communication interface may allow the processors 220 of the system 400 to receive and transmit the various data of the system 400 therebetween.

Though some embodiments may mention a single computing entity 200 of a user 405, one with skill in the art will recognize that multiple computing entities 200 of multiple users 405 may be used without departing from the inventive subject matter described herein. Additionally, though some embodiments may refer to a single display 316 and/or single medical charging/fueling station 600, one with skill in the art will recognize that multiple displays 316 and/or medical charging/fueling station 600s may be linked together in a way that creates a “single” display 316 and/or medical charging/fueling station 600 that may be used in a manner not departing from the inventive subject matter described herein. For instance, four OLED televisions may be linked together in way that creates a multi-display that the system 400 may use as a “single” display 316. For instance, two or more charging/fueling stations may be connected to a shell, wherein users 405 may park about said shell containing said charging/fueling stations so that they may charge/fuel their vehicle as well as access displays 316 incorporated into said charging/fueling stations. Additionally, one with skill in the art will recognize that a plurality of displays 316 may be controlled by a single control board, and the single control board may manage the plurality of display windows 317 about the display user interfaces 316A of the plurality of displays 316. In yet another preferred embodiment, two or more control boards of two or more displays 316 may be operably connected to one another and manage the plurality of display windows 317 about the display user interfaces 316A of the plurality of displays 316 in collaboration with one another. Accordingly, one with skill in the art will recognize that displays 316 may be used in combination with one or more control boards and one or more computing entities 200 in a number of ways without departing from the inventive subject matter described herein.

Generally, the system 400 is designed to identify users 405 of the system 400 and allow users 405 of the system 400 to undergo a medical checkup. Users 405 may operably connect to displays 316 of a medical charging/fueling station 600 via computing entities 200 and select data to be presented within a display user interface 316A of the display 316 of the medical charging/fueling station 600 and/or a display 316 within their vehicle. In a preferred embodiment, a patient user may communicate with a healthcare professional user via the system 400, wherein the users 405 may manipulate their computing entities 200 to control information presented on displays 316 in a way that allows the users 405 to interact with one another. For instance, a user 405 of a medical charging/fueling station 600 may use a secondary security means to associate a first computing entity 200 of the user 405 with a first display 316 of the medical charging/fueling station 600. The user 405 may then manipulate the user interface 411 of the first computing entity 200 in a way that connects the first display 316 of the medical charging/fueling station 600 to a second display 316 of a healthcare professional, wherein a computing entity 200 of the healthcare professional is operably connected to the second display 316. The user 405 of the medical charging/fueling station 600 may choose user content 815, 835, 855 to present within the display windows 317 of the display user interface 316A of the first display 316, and the user content 815, 835, 855 chosen by the user 405 of the medical charging/fueling station 600 may also be presented to the healthcare professional via the plurality of display windows 317 of the display user interface 316A of the second display 316. Alternatively, the healthcare professional may choose user content 815, 835, 855 to present within the display windows 317 of the display user interface 316A of the second display 316, and the user content 815, 835, 855 chosen by the healthcare professional may also be presented to the user 405 of the medical charging/fueling station 600 via the plurality of display windows 317 of the display user interface 316A of the first display 316. Content chosen by the users 405 to be presented within the plurality of display windows 317 preferably comprises image data 430B.

In a preferred embodiment, a control board 409 of a display 316 receives user data 430A, image data 430B, application data 430C, patient data 430D, and display data 430E from a computing entity 200 200 and/or medical device 407. The control board 409 may then present said user data 430A, image data 430B, application data 430C, patient data 430D, and display data 430E via the display 316 in the display user interface 316A. In another preferred embodiment, the display 316 may be configured to receive user data 430A, image data 430B, application data 430C, patient data 430D, and display data 430E via a server 110 and/or database 115 when selected by a user 405 via the user interface 411 of the computing entity 200 and/or the display user interface 316A of the display 316. In a preferred embodiment, the user data 430A, image data 430B, application data 430C, patient data 430D, and display data 430E is streamed/mirrored from the computing entity 200 200, server 110, and/or database 115 to the control board 409, wherein the control board 409 inserts said streamed/mirrored patient data 430D into the display user interface 316A. Alternatively, the control board 409 may manipulate the patient data 430D and/or display user interface 316A based on commands received from an input device. In one preferred embodiment, the display user interface 316A may also comprise a control window, which may allow a user 405 to control the layout of the display user interface 316A. For instance, a user 405 may choose a layout that separates the display user interface 316A into multiple windows arranged in a particular way. In some embodiments, the control window may allow a user 405 to alter the size and orientation of a display window 317 of the display user interface 316A. Alternatively, an input device having a plurality of layouts thereon may be used to manipulate the layout of the display user interface 316A. The input device may be connected to the system 400 via a wired or wireless connection. In a preferred embodiment, the input device transmits a computer readable signal containing instructions to the control board 409, which the control board 409 uses to manipulate data presented via the display user interface 316A.

In a preferred embodiment, a user 405 logs into a user profile 430 of the system 400 before accessing the various features of a display 316, allowing the system 400 to verify the identity of the user 405. A user interface 411 of a computing entity 200 allows a user 405 to input login credentials and/or commands. A processor 220 operably connected to said computing entity 200 and said display 316 sends the login credentials and/or commands to a control board of said display 316 via a computer readable signal, wherein said login credentials and/or commands of said computer readable signal allow access to said display 316 should they be associated with a user profile 430 having sufficient permission levels. A user 405 may then manipulate the user interface of the computing entity 200 in a way that allows said user 405 to choose various data of the system 400 to be presented on the display 316 for review. In some preferred embodiments, a user 405 may be required to use a secondary security method to access a display 316 to present the various data of the system 400. For instance, a user 405 may be required to use a camera of their computing entity 200 to scan a predefined pattern, such as a bar code or a QR code, that is presented on a display 316, which may associate that user 405 with a particular display 316.

In a preferred embodiment, displays 316 of the system 400 are configured for remote communication. Preferably, a first user uses a secondary security method to link a first computing entity 200 to a first display 316 and second user uses a secondary security method to link a second computing entity 200 to a second display 316. Once connected, the users 405 may select the various data of the system 400 which they would like to be presented within the plurality of display windows 317 of the displays 316. For instance, a patient located in a rural area may use a secondary security method to associate a display 316 of a local medical charging/fueling station 600 with their computing entity 200 and user profile 430. One or more healthcare professionals within a medical facility may be logged into a second display 316 of the system 400 that is operably connected to the first display 316, allowing said one or more healthcare professionals to select various data of the system 400 to be presented within the display windows 317 of the display user interface 316A. The displays 316 are preferably operably connected to one another in a way such that data presented within the display windows 317 of each display 316 is the same. However, though the same data may be presented within the display windows 317 of operably connected displays 316, the display windows 317 may or may not be organized in the same manner within the display user interfaces 316A of the displays 316. In a preferred embodiment, each control board of a display 316 controls how the user content 815, 835, 855 is organized within display windows 317 of the display user interface 316A.

Medical devices 407 that may be operably connected to the computing entity 200 and/or display 316 include, but are not limited to, heart rate monitors, electrocardiograms, pulse oximeters, blood pressure meters, blood glucose meters, smart watches, or any combination thereof. In one preferred embodiment, the medical devices 407 are immovably secured to the display 316. For instance, a medical charging/fueling station 600 may comprise a blood pressure meter and electrocardiograms device that are immovably attached thereto. A user 405 who has seated themselves within an examination area of a medical charging/fueling station 600 may position their arm within a cuff of the blood pressure meter and grip EKG electrodes in a way such that the user's blood pressure, heart rate, and heart rhythm may be measured and saved by the system 400 as patient data 430D. In other preferred embodiments, medical devices 407 may be moveably attached to the medical charging/fueling station 600. For instance, a pulse oximeter within a privacy room 607 connected to the medical charging/fueling station 600 may be moveably secured to the medical charging/fueling station 600 in a way such that a user 405 may remove said pulse oximeter from a mount of said medical charging/fueling station 600 so that blood oxygen data may be collected and saved in the form of patient data 430D. In yet another preferred embodiment, medical devices 407 may be configured to operably connect to the display 316 and/or computing entity 200. For instance, a user's smart watch configured to collect patient data 430D may be used by the system 400 to inform a medical professional as to the health of the user 405, as illustrated in FIG. 6C. Accordingly, medical devices 407 may be connected to the display 316 in a plurality of ways without departing from the inventive subject matter as described herein.

In some preferred embodiments, the system 400 may further comprise a secondary security device. Devices that may act as the secondary security device may include, but are not limited to, biometric devices, key cards, wearables, or any combination thereof. In a preferred embodiment, devices that may act as the biometric devices include, but are not limited to, contact biometric devices, such as fingerprint scanners and hand geometry scanners, and/or non-contact biometric devices, such as face scanners, iris scanners, retina scanners, palm vein scanners, and voice identification devices. In some embodiments, the secondary security device may be operably connected to the computing entity 200 and/or display 316 in a way such that it is in direct communication with the computing entity 200 and/or display 316 and no other computing entity 200 and/or display 316. For instance, the secondary security device in the form of a facial recognition camera may be securely and directly connected to a control board 409 of the display 316 such that a user 405 must biometrically scan their face prior to the system 400 allowing access to the various data and features of the system 400. In some preferred embodiments, biometric data associated with a user 405 is saved in a user profile 430 as user data 430A, which the system 400 uses to verify a user's identity. For instance, secondary security devices may be securely and directly connected to a first computing entity 200 and a second computing entity 200 in a way such that both a first user of the first computing entity 200 and a second user of the second computing entity 200 must biometrically scan thumbprints prior to the system 400 allowing the first user and second user to access the various data of the system 400.

In a preferred embodiment, key cards and wearables preferably comprise a secure transmitter configured to transmit login credentials to the computing entity 200 and/or control board of the display 316. Wearables having a secure transmitter include clothing and accessories, such as shirts, pants, jackets, belts, shoes, wristbands, watches, glasses, pins, nametags, etc., that have said transmitter attached thereto and/or incorporated therein. The secure transmitter preferably contains login credentials in the form of a unique ID, which may be conveyed to a computing entity 200 and/or control board of a display 316 in the form of a computer readable signal. Unique IDs contained within the computer readable signal that has been broadcast by the transmitter may include, but are not limited to, unique identifier codes, social security numbers, personal identification numbers (PINs), etc. For instance, a computer readable signal broadcast by a secondary security device in the form of a wrist band may contain information that will alert the control board of the display 316 that a particular user is within a certain range, which may cause the system 400 to allow a user 405 to access the various data of the system 400 if additional steps are taken.

Types of devices that may act as the transmitter include, but are not limited, to near field communication (NFC), Bluetooth, infrared (IR), radio-frequency communication (RFC), radio-frequency identification (RFID), and ANT+, or any combination thereof. In an embodiment, transmitters may broadcast signals of more than one type. For instance, a transmitter comprising an IR transmitter and RFID transmitter may broadcast IR signals and RFID signals. Alternatively, a transmitter may broadcast signals of only one type of signal. For instance, identification (ID) cards may be fitted with transmitters that broadcast NFC signals containing unique IDs associated with a particular user, wherein displays 316 equipped with NFC receivers must receive said NFC signals containing unique IDs before access to one or more features of the display user interface 316A may be granted.

Use of secondary security devices may be used solely or in addition to secondary security methods of the system 400, allowing the system 400 to have flexible multifactor identification. Simultaneous use may be beneficial to prevent unauthorized access of the various data of the system 400 and/or communications between a user 405 and a healthcare professional. For instance, the system 400 may require that a user 405 use both a secondary security method and biometric scanner for identification purposes before allowing a user 405 to access the various features of the system 400. In another preferred embodiment, the system 400 may use a secondary security method for identification purposes and a key card for activating other features of the system 400. For instance, a user 405 may cause the display 316 of the medical charging/fueling station 600 to enter a healthcare mode by scanning a key card having a secure transmitter. The user 405 may then use a secondary security method to allow the system 400 to identify the user 405 and associate a computing entity 200 of the user 405 with the display 316 of the vehicle. Medical devices 407 of the user 405 may transmit patient data 430D to the display 316 and/or computing entity 200 where it may be used by the system 400 to perform the various functions described herein.

In a preferred embodiment, the various data of the system 400 may be stored in user profiles 430. In a preferred embodiment, a user profile 430 is related to a particular user. A user 405 is preferably associated with a particular user profile 430 based on a username. However, it is understood that a user 405 may be associated with a user profile 430 using a variety of methods without departing from the inventive subject matter herein. Types of data that may be stored within user profiles 430 of the system 400 include, but are not limited to, user data 430A, image data 430B, application data 430C, patient data 430D, and display data 430E. Some preferred embodiments of the system 400 may comprise a database 115 operably connected to the processor 220. The database 115 may be configured to store user data 430A, image data 430B, application data 430C, patient data 430D, and display data 430E within user profiles 430 and/or separately. As used herein, user data 430A may be defined as personal information of a user 405 that helps the system 400 identify the user 405 and their interests. Types of data that may be used by the system 400 as user data 430A includes, but is not limited to, a user's name, username, social security number, phone number, email address, physical address, gender, age, or any combination thereof.

As used herein, image data 430B may be defined as photographic or trace objects that represent the underlying pixel data of an area of an image element, which is created, collected, and stored using image constructor devices, such as a camera. For instance, the system 400 may use image data 430B obtained via a scanning device and/or a secondary security device to confirm the identity of a user 405. For instance, image data 430B of patient data 430D may be transmitted to the display 316 and presented to the user 405 where it may be reviewed/manipulated by a medical professional to determine the health of the user 405. For instance, image data 430B of a display application may be transmitted to the display 316 from the computing entity 200, server 110, and/or database 115 where it may be manipulated by the control board within the plurality of display windows 317 of the display user interface 316A. As used herein, patient data 430D may be defined as information related to a user's medical record, which may usually be found within an electronic health record. Types of data that may be used by the system 400 as patient data 430D includes, but is not limited to, encounter notes, lab/image reports, orders, medications, guidelines, assessments, interventions, pathological reports, or any combination thereof.

Application data 430C may be defined as instructions that cause a display application of the display 316 to perform an action. In one preferred embodiment, the system 400 may determine whether a user application of the computing entity 200 is compatible with a display application of the display 316. If it is determined that the display application and user application are compatible, application data 430C may be transmitted to the display 316 from the computing entity 200 in lieu of image data 430B. The display application is controlled by the control board of the display 316 and inserted into a display window 317 of the display user interface 316A. Instructions input into a compatible user application are transmitted to the control board from the computing entity 200 and are used by the control board to perform actions of the display application, reducing the amount of data transferred between the computing entity 200 and display 316. For instance, a medical device 407 operably connected to the computing entity 200 may transmit patient data 430D to a user application version of a health application of the computing entity 200. A display application version of the health application and the user application version of said health application may be compatible in a way such that a user 405 may open the user application version on their computing entity 200 and subsequently instruct the system 400 (via the user interface 411) to present the user application version in a display window 317 of the display user interface 316A.

The processor 220 of the control board may then determine if the display application version of the health application is compatible with the user application version of the health application. If the display application version and user application version are compatible, the control board may open the display application version of the health application locally and manipulate it via instructions received from the computing entity 200 as patient data 430D is received or actions are taken via the user application version. If the display application version and user application version are not compatible, the control board may receive image data 430B of the user application version and present it within a display window 317 of the display user interface 316A. As used herein, display data 430E may be defined as data that may be used to identify a particular display 316 of the system 400. Display data 430E may include, but is not limited to, geolocation data, display name, display descriptions, or any combination thereof. User data 430A, patient data 430D, and image data 430B in combination with permission levels 800 is used by the system 400 to assist in presenting patient data 430D to users 405 within the display user interface 316A.

As previously mentioned, some preferred embodiments of the display 316 may further comprise a control board 409. The control board 409 comprises at least one circuit and microchip. In another preferred embodiment, the control board 409 may further comprise a wireless communication interface, which may allow the control board 409 to receive instructions from an input device controlled by a user 405. In a preferred embodiment, the control board 409 may control the plurality of display windows 317 of the display user interface 316A and the patient data 430D presented therein. The microchip of the control board 409 comprises a microprocessor and memory. In another preferred embodiment, the microchip may further comprise a wireless communication interface in the form of an antenna. The microprocessor may be defined as a multipurpose, clock driven, register based, digital-integrated circuit which accepts binary data as input, processes it according to instructions stored in its memory, and provides results as output. In a preferred embodiment, the microprocessor may receive patient data 430D from a server 110 and/or database 115 via the wireless communication interface, wherein the patient data 430D comprises image data 430B in the form of a video.

As mentioned previously, the system 400 may comprise a user interface 411. A user interface 411 may be defined as a space where interactions between a user 405 and the system 400 may take place. In an embodiment, the interactions may take place in a way such that a user 405 may control the operations of the system 400. A user interface 411 may include, but is not limited to operating systems, command line user interfaces, conversational interfaces, web-based user interfaces, zooming user interfaces, touch screens, task-based user interfaces, touch user interfaces, text-based user interfaces, intelligent user interfaces, brain-computer interfaces (BCIs), and graphical user interfaces, or any combination thereof. The system 400 may present data of the user interface 411 to the user 405 via a display 316 operably connected to the processor 220. A display 316 may be defined as an output device that communicates data that may include, but is not limited to, visual, auditory, cutaneous, kinesthetic, olfactory, and gustatory, or any combination thereof.

In a preferred embodiment, the control board 409 of the display 316 receives data of the system 400 from the server 110 and/or database 115 and may then present said data via at least one display window 317 of a display 316 of a medical charging/fueling station 600. In one preferred embodiment, the data is streamed from the database 115 and/or server 110 to the control board 409, wherein the control board 409 inserts said streamed data into said at least one display window 317. In another preferred embodiment, image data 430B is transmitted to the display 316 of the medical charging/fueling station 600 from the computing entity 200. The control board 409 may automatically select a layout of the display user interface 316A, wherein said layout may be determined based on a plurality of variables, including, but not limited to, number of users, type of content being viewed by the user(s), user preferences, user location, or any combination thereof. For instance, the control board 409 may select a layout for the display user interface 316A that presents a telehealth conference of the user 405 while simultaneously displaying information pertaining to the charging/fueling of a vehicle to which the medical charging/fueling station 600 is connected. For instance, the control board 409 may select a layout for the display user interface 316A that presents patient data 430D collected by a medical device 407 of the medical charging/fueling station 600 in a first display window, analysis of said patient data 430D by a machine learning technique 450 of the medical charging/fueling station 600 in a second window, and a social media application of the computing entity 200 of the user 405 in a third window.

Information presented via a display 316 may be referred to as a soft copy of the information because the information exists electronically and is presented for a temporary period of time. Information stored on the non-transitory computer-readable medium 416 may be referred to as the hard copy of the information. For instance, a display 316 may present a soft copy of visual information via a liquid crystal display (LCD), wherein the hardcopy of the visual information is stored on a local hard drive. For instance, a display 316 may present a soft copy of audio information via a speaker, wherein the hard copy of the audio information is stored in RAM. For instance, a display 316 may present a soft copy of tactile information via a haptic suit, wherein the hard copy of the tactile information is stored within a database 115. Displays 316 may include, but are not limited to, cathode ray tube monitors, LCD monitors, light emitting diode (LED) monitors, gas plasma monitors, screen readers, speech synthesizers, haptic feedback equipment, virtual reality headsets, speakers, and scent generating devices, or any combination thereof.

The database 115 may be operably connected to the processor 220 via wired or wireless connection. In a preferred embodiment, the database 115 is configured to store user data 430A, image data 430B, application data 430C, patient data 430D, and display data 430E within user profiles 430. Alternatively, the user data 430A, image data 430B, application data 430C, patient data 430D, and display data 430E stored within user profiles 430 may be stored on the non-transitory computer-readable medium 416. The database 115 may be a relational database such that the user data 430A, image data 430B, application data 430C, patient data 430D, and display data 430E associated with each user profile 430 and within the plurality of user profiles 430 may be stored, at least in part, in one or more tables. Alternatively, the database 115 may be an object database such that user data 430A, image data 430B, application data 430C, patient data 430D, and display data 430E associated with each user profile 430 of the plurality of user profiles 430 may be stored, at least in part, as objects. In some instances, the database 115 may comprise a relational and/or object database and a server 110 dedicated solely to managing the user data 430A, image data 430B, application data 430C, patient data 430D, and display data 430E in the manners disclosed herein.

As illustrated in FIG. 7, the system 400 may comprise a medical charging/fueling station 600 having a display 316, which may be used to for virtual medical checkups. In a preferred embodiment, a medical charging/fueling station 600 may comprise a charging meter 605, charging plug 610, at least one medical measuring device and display 316, wherein a plurality of display windows 317 may be used to provide patient data 430D to a user 405 as well as to facilitate communication between a user 405 and a healthcare professional. The charging meter 605 is configured to obtain payment information from a user 405 and/or obtain user data 430A. In an alternative embodiment, the charging meter may be used to facilitate communication for and payment of medical services that are to be received by a willing user 405. In a preferred embodiment, the charging meter 605 may provide the user 405 with information pertaining to the charge of an electric vehicle battery/fuel cell. The charging plug 610 is configured to fit within a charging outlet of an electric vehicle 705 in order to charge a battery/fuel cell of said electric vehicle 705. However, other types of fueling/charging systems may be used without departing from the inventive subject matter described herein. For instance, some embodiments may comprise traditional gas pumps or pumps that disperse chemicals designed to regenerate fuel cells.

In one preferred embodiment, the system 400 may obtain the user data 430A of a user 405 via a wireless connection when the user 405 is within a certain range of the medical charging/fueling station 600. The user data 430A may be used by the medical charging/fueling station 600 to identify a particular user, which preferably occurs when payment information is given to the medical charging/fueling station 600 by the user 405. In some preferred embodiments, this user data 430A may then be used by the system 400 to present targeted advertising to the user 405 via a display 316 within the user's vehicle and/or a display 316 of the medical charging/fueling station 600. In another preferred embodiment, a user 405 may activate a medical charging/fueling station 600 via a user interface 411 of the user's computing entity 200, wherein activation of the medical charging/fueling station 600 would cause the medical charging/fueling station 600 to receive the user data 430A of the user profile 430 of the user 405, allowing for the facilitation of a virtual medical checkup. In some preferred embodiments, as illustrated in FIG. 7, an energy station comprising a plurality of medical charging/fueling station 600 may be used as a hub for providing targeted advertising to a plurality of users 405.

In some preferred embodiments, the user interface 411 and/or display user interface 316A may comprise additional controls that allow users 405 of the system 400 to manipulate how the various data of the system 400 is presented within the display windows 317. In a preferred embodiment, access to these features is based on permission levels 800 of the user 405. For instance, the system 400 may be configured in a way such that a user 405 may only choose certain information to be presented within the display windows 317 of the display user interface 316A should they have the appropriate permissions within their user profile 430. For instance, the system 400 may be configured in a way such that a user 405 may only alter the arrangement of display windows 317 about the display user interface 316A should they have appropriate permissions within their user profile 430. For instance, the system 400 may be configured in a way such that display windows 317 containing particular data may be presented to a first user having appropriate permissions via a first display 316 but not to a second user of a second display 316 not having appropriate permissions. Accordingly, one with skill in the art will understand that user data 430A, image data 430B, application data 430C, patient data 430D, and display data 430E may be used by the system 400 multiple ways to carry out various functions of the system 400 without departing from the inventive subject matter described herein.

In another preferred embodiment, the system 400 may be used for communication with emergency personnel services, including 911 call centers, police officers, firefighters, and emergency management agencies. For instance, a user 405 may use emergency functionality of the display user interface 316A in a way that allows the user to quickly contact emergency personnel services. Once emergency functionality is activated, the processor may transmit a computer readable signal to emergency personnel services and connect the user 405 with a representative thereof, reducing the e time to report an emergency. In a preferred embodiment, display data 430E containing the location in which the emergency call has been initiated is transmitted to an emergency dispatch center, ensuring that emergency personnel services are directed to the exact location of the emergency without delay. This feature is particularly useful in situations in which a user is unfamiliar with an area or in rural areas where a precise location may be more difficult to describe.

In embodiments where a user activates emergency functionality while undergoing a medical emergency, the system's capability to collect patient data may provide emergency responders with important pre-arrival information. For instance, a user experiencing symptoms of a heart attack may use an operably connected medical device to relay real-time data—such as heart rate, blood pressure, and electrocardiogram readings—to the EMTs en route. This data allows emergency personnel to prepare the appropriate medical equipment and treatment strategies before arriving on the scene, potentially improving patient outcomes. In embodiments comprising machine learning techniques, the data may be analyzed via said machine learning techniques and recommend appropriate actions to be taken by EMTs upon their arrival.

In embodiments where a user activates emergency functionality to report something other than a medical emergency, emergency personnel services responding to incidents at or near medical charging/fueling stations may be able use the system 400 as a secure communication hub or information source. For instance, the system's displays 316 may be used by police officers to provide live video feeds, maps, and other situational awareness tools to assist in coordinating response efforts. For instance, the system 400 may be configured to access vehicle information, such as whether the EV is actively charging, during a fire at a medical charging/fueling station. In some preferred embodiments, the system 400 may be configured to allow emergency personnel services to control features of the system 400 after identification via a secondary security device. In some preferred embodiments, the system 400 may grant temporary access to system data to emergency responders, allowing the emergency responders to view pertinent medical data or vehicle information.

In a preferred embodiment, the system 400 is integrated with existing emergency response protocols to enhance the efficiency of emergency services. For instance, the system 400 may be programmed to recognize specific emergency codes and automatically provide relevant information to responders, such as the nearest hospital with an available trauma center or the location of the closest fire hydrant. For instance, during severe weather events, the system 400 may be programmed to provide information to a user as to a particular route to take to avoid said severe weather event and/or actions that should be taken by the user should they find themselves in danger due to said severe weather event. Accordingly, the system 400 may be used in multiple ways with emergency infrastructure to inform users of emergencies and provide emergency responders with immediate, accurate, and comprehensive information pertaining to emergencies in order to allow said emergency responders to make informed decisions and take swift action to protect and save lives.

The system 400 preferably uses artificial intelligence (AI) techniques to perform functions of the system 400. In one preferred embodiment, AI techniques may be used to control the number of display windows 317 presented within the display user interface 316A. In another preferred embodiment, AI techniques may be used to organize the plurality of display windows 317 within the display user interface 316A. In yet another preferred embodiment, AI techniques may be used to evaluate patient data 430D collected by the system 400, wherein said evaluations may be used to assist medical professionals in a separate evaluation of patients. In yet another preferred embodiment, AI techniques may be used by the system 400 to determine when a user 405 is experiencing a medical emergency or may be a driving risk. The term “artificial intelligence” and grammatical equivalents thereof are used herein to mean an intelligence method used by the system 400 to correctly interpret and learn from data of the system 400 or a plurality of systems in order to achieve specific goals and tasks through flexible adaptation. Types of intelligence methods that may be used by the system 400 include, but are not limited to, machine learning, neural network, computer vision, or any combination thereof.

The system 400 preferably uses machine learning techniques 450 to perform the methods disclosed herein, wherein the instructions carried out by the processor 220 for said machine learning techniques 450 are stored on the non-transitory computer-readable medium 416, server 110, and/or database 115. Machine learning techniques 450 that may be used by the system 400 include, but are not limited to, classification algorithms, neural network algorithm, regression algorithms, decision tree algorithms, clustering algorithms, genetic algorithms, supervised learning algorithms, semi-supervised learning algorithms, unsupervised learning algorithms, deep learning algorithms, or other types of algorithms. More specifically, machine learning algorithms can include implementations of one or more of the following algorithms: support vector machine, decision tree, nearest neighbor algorithm, random forest, ridge regression, Lasso algorithm, k-means clustering algorithm, boosting algorithm, spectral clustering algorithm, mean shift clustering algorithm, non-negative matrix factorization algorithm, elastic net algorithm, Bayesian classifier algorithm, RANSAC algorithm, orthogonal matching pursuit algorithm, bootstrap aggregating, temporal difference learning, backpropagation, online machine learning, Q-learning, stochastic gradient descent, least squares regression, logistic regression, ordinary least squares regression (OLSR), linear regression, stepwise regression, multivariate adaptive regression splines (MARS), locally estimated scatterplot smoothing (LOESS) ensemble methods, clustering algorithms, centroid based algorithms, principal component analysis (PCA), singular value decomposition, independent component analysis, k nearest neighbors (kNN), learning vector quantization (LVQ), self-organizing map (SOM), locally weighted learning (LWL), apriori algorithms, eclat algorithms, regularization algorithms, ridge regression, least absolute shrinkage and selection operator (LASSO), elastic net, classification and regression tree (CART), iterative dichotomiser 3 (ID3), C4.5 and C5.0, chi-squared automatic interaction detection (CHAID), decision stump, M5, conditional decision trees, least-angle regression (LARS), naive bayes, gaussian naïve bayes, multinomial naïve bayes, averaged one-dependence estimators (AODE), bayesian belief network (BBN), bayesian network (BN), k-medians, expectation maximisation (EM), hierarchical clustering, perceptron back-propagation, hopfield network, radial basis function network (RBFN), deep boltzmann machine (DBM), deep belief networks (DBN), convolutional neural network (CNN), stacked auto-encoders, principal component regression (PCR), partial least squares regression (PLSR), sammon mapping, multidimensional scaling (MDS), projection pursuit, linear discriminant analysis (LDA), mixture discriminant analysis (MDA), quadratic discriminant analysis (QDA), flexible discriminant analysis (FDA), bootstrapped aggregation (bagging), adaboost, stacked generalization (blending), gradient boosting machines (GBM), gradient boosted regression trees (GBRT), random forest, or even algorithms yet to be invented.

In a preferred embodiment, the system 400 may monitor patient data 430D of a user 405 and processes said data using a machine learning technique 450 to determine potential medical issues a user 405 may be experiencing. For instance, the system 400 may obtain audio data from a user 405 and process it using natural language processing (NLP) to discern what symptoms a user 405 believes they have been experiencing in their own words. The system 400 may then use semi-supervised learning to diagnose a user 405 with an illness. In some preferred embodiments, the system 400 may use semi-supervised learning to create a treatment protocol for the illness that was previously determined. In one preferred embodiment, the system 400 may continuously monitor patient data 430D of a user 405 who is determined to have an illness and use semi-supervised learning to adjust the treatment protocol of said illness based on changes in said patient data 430D over time. In one preferred embodiment, the system 400 may use machine learning techniques 450 to assist a healthcare professional with diagnosing an illness. For instance, a user 405 having a plurality of symptoms indicative of an upper respiratory infection may use the system 400 to interact with a nurse practitioner. The system 400 may obtain patient data 430D from the patient and from the EHR, which it may subsequently analyze via a decision tree, supervised learning to assist the nurse practitioner in determining the most likely respiratory illnesses. A user 405 may then go to a rapid testing facility where the user 405 may undergo rapid testing for the designated upper respiratory illnesses. In some preferred embodiments, the machine learning technique 450 may also assist the nurse practitioner in prescribing medication to a user 405 based on a determined illness.

In some preferred embodiments, patient data 430D collected and processed by the system 400 may be monitored using a machine learning technique 450 to determine when the user 405 may be experiencing a medical emergency and subsequently call emergency medical services to assist the user 405. For instance, the system 400 may be operably connected to a medical device 407 configured to measure blood sugar of a user 405. Based on a user's blood sugar as measured by the medical device 407, the system 400 may use unsupervised learning to determine that a user's blood sugar is at a dangerously low or high level and subsequently call emergency medical services to assist the user 405. For instance, an electrocardiogram and heart rate monitor operably connected to the medical charging/fueling station 600 may collect patient data 430D related to the heart of the driver. Based on symptoms as described by the user 405 and additional symptoms as determined by the system 400 through natural language processing, the system 400 may determine that the user 405 is experiencing a heart related medical emergency and alert emergency medical services of the user's description, geolocation based on the display data 430E, and the nature of the medical emergency.

In a preferred embodiment, the system 400 may use more than one machine learning technique 450 to monitor patient data 430D of a user 405. For instance, the system 400 comprising a microphone may use a combination of NLP and reinforcement learning to discern symptoms, such as congestion, hoarseness, and shortness of breath, which may be discerned by the system in addition to symptoms described by said patient. If the system 400 determines that a user 405 has additional symptoms not described by the patient, the system 400 may add those symptoms to a list of symptoms before making an evaluation of the patient data 430D or relaying said symptoms to a medical professional. In another preferred embodiment, the system 400 may actively monitor a user's alertness to determine if a user 405 is coherent enough to make decisions. For instance, the system 400 comprising a camera may use a combination of facial emotion recognition (FER) and deep learning to discern the sobriety of a user 405. Should the system 400 determine that a user 405 is below a sobriety threshold, the system 400 may recommend that the user 405 not operate a vehicle and/or alert emergency personnel services. In another preferred embodiment, should the system 400 determine that a user 405 is dangerously intoxicated, the system 400 may alert emergency personnel to assist said user 405.

In a preferred embodiment, the machine learning techniques 450 comprise instructions configured to create a trained machine learning techniques 450 from at least some training data and according to an implementation of the machine learning techniques 450, wherein the training data serves as a baseline dataset that may act as the foundational data of the machine learning techniques 450. The instructions of the machine learning techniques 450 dictate how the machine learning techniques 450 gain knowledge from the various data sources of the system 400 and may comprise various types of programable instructions that include, but are not limited to, local commands, remote commands, executable files, protocol commands, selected commands, or any combination thereof. The instructions of the machine learning techniques 450 may vary widely, depending on a desired implementation. In a preferred embodiment, instructions may include streamed-lined instructions that instruct the machine learning techniques 450 on how to train the system 400, possibly in the form of a script (e.g., Python, Ruby, JavaScript, etc.). In another preferred embodiment, the instructions may include data filters or data selection criteria that define requirements for desired results sets created from the various data of the system 400 as well as which machine learning algorithm is to be used.

Training of the machine learning techniques 450 may be supervised, semi-supervised, or unsupervised. In some preferred embodiments, the machine learning techniques 450 may use NLP to analyze data (e.g., audio data, text data, etc.) that may be used to train the machine learning techniques 450. For instance, the system 400 may use natural language processing and deep learning to ascertain a baseline voice of a user 405 when alert, which may be used by the system 400 to determine when a user 405 may be feeling tired. Training of the machine learning techniques 450 may result in baseline machine learning techniques 450 that may serve as AI techniques for performing the various functions of the system 400 in the manners described herein. Baseline machine learning techniques 450 may further be configured to act as passive models or active models. A passive model may be described as a final, completed machine learning model that uses only the baseline data set to establish behavior of the baseline machine learning technique 450. An active model may be described as a plasticity machine learning model that is dynamic in that it may be updated using both the baseline dataset and data outside of the baseline data set.

In a preferred embodiment, the system 400 may use a passive model to allow for a high degree of control as to how the system 400 manages user interfaces and display windows 317 in the manners described herein. For instance, a passive model may be configured via a private dataset to provide each user 405 of the system 400 with the same driving recommendations. These recommendations may be made by the system 400 regardless of user data 430A that may indicate that particular users have historically preferred certain actions in certain circumstances. A passive model may be especially useful for users 405 having user profiles 430 with little user data 430A from which the machine learning techniques 450 may learn from. In some preferred embodiments, the system 400 may be configured to begin as passive models until a threshold amount of user data 430A has been acquired. Once the threshold amount of user data 430A has been acquired, the system 400 may cause the machine learning techniques 450 to switch to active models, allowing the system 400 to make recommendations to a user 405 that better parallel historical preferences of the user 405. For instance, a system 400 may be configured to organize display windows 317 based on a passive model for the first ten times a particular healthcare professional uses the system 400 to analyze patient data 430D of a patient user, wherein the system 400 may also be configured to determine display window location preferences of a user 405 after the display windows 317 are automatically organized within a display user interface 316A by the system 400. After the system 400 has organized the display windows 317 ten times, the machine learning techniques 450 of the system 400 may switch to an active machine model for that particular user and organize display windows 317 based on the actions of the user 405 after the display windows 317 were automatically organized within a display user interface 316A by the system 400.

In some embodiments, an active machine model may be updated in real-time, daily, weekly, bimonthly, monthly, quarterly, or annually using the various data (e.g., to update model instructions, shifts in time, new/corrected private data sets, user data 430A, patient data 430D, etc.) of the system 400. In some preferred embodiments, the passive machine model may also be updated as new/updated private data sets become available. In a preferred embodiment, machine learning techniques 450 comprise metadata that describe the state of the passive/active model with respect to its updates. The metadata may include attributes describing one or more of the following: a version number, date updated, amount of new data used for the update, shifts in model parameters, convergence requirements, or other information. Because each user 405 of the system 400 may potentially have a unique machine learning technique 450 associated with their user profile 430 due to the personal nature of user data 430A associated with each user profile 430, such information allows for identifying distinct passive/active models within the system 400 that may be separately managed.

To prevent an un-authorized user from accessing other user's information, the system 400 may employ a security method. As illustrated in FIG. 8, the security method of the system 400 may comprise a plurality of permission levels 800 that may grant users 405 access to user content 815, 835, 855 within the database 115 while simultaneously denying users 405 without appropriate permission levels 800 the ability to view user content 815, 835, 855. To access the user content 815, 835, 855 stored within the database 115, users 405 may be required to make a request via a user interface 411. Access to the data within the database 115 may be granted or denied by the processor 220 based on verification of a requesting user's 805, 825, 845 permission level 800. If the requesting user's 805, 825, 845 permission level 800 is sufficient, the processor 220 may provide the requesting user 805, 825, 845 access to user content 815, 835, 855 stored within the database 115. Conversely, if the requesting user's 805, 825, 845 permission level 800 is insufficient, the processor 220 may deny the requesting user 805, 825, 845 access to user content 815, 835, 855 stored within the database 115. In an embodiment, permission levels 800 may be based on user roles 810, 830, 850 and administrator roles 870, as illustrated in FIG. 8. User roles 810, 830, 850 allow requesting users 805, 825, 845 to access user content 815, 835, 855 that a user 405 has uploaded and/or otherwise obtained through use of the system 400. Administrator roles 870 allow administrators 865 to access system 400 wide data.

In an embodiment, user roles 810, 830, 850 may be assigned to a user 405 in a way such that a requesting user 805, 825, 845 may view user profiles 430 containing user data 430A, image data 430B, application data 430C, patient data 430D, and display data 430E via a user interface 411. To access the data within the database 115, a user 405 may make a user request via the user interface 411 to the processor 220. In an embodiment, the processor 220 may grant or deny the request based on the permission level 800 associated with the requesting user 805, 825, 845. Only users 405 having appropriate user roles 810, 830, 850 or administrator roles 870 may access the data within the user profiles 430. For instance, as illustrated in FIG. 8, requesting user 1 805 has permission to view user 1 content 815 and user 2 content 835 whereas requesting user 2 825 only has permission to view user 2 content 835. Alternatively, user content 815, 835, 855 may be restricted in a way such that a user may only view a limited amount of user content 815, 835, 855. For instance, requesting user 3 845 may be granted a permission level 800 that only allows them to view user 3 content 855 related to their specific interest but not user 3 content 855 related to the identity of said user 405. In the example illustrated in FIG. 8, an administrator 865 may bestow a new permission level 800 on users 405 so that it may grant them greater permissions or lesser permissions. For instance, an administrator 865 may bestow a greater permission level 800 on other users 405 so that they may view user 3's content 855 and/or any other user's content 815, 835, 855. Therefore, the permission levels 800 of the system 400 may be assigned to users 405 in various ways without departing from the inventive subject matter described herein.

The subject matter described herein may be embodied in systems, apparati, methods, and/or articles depending on the desired configuration. In particular, various implementations of the subject matter described herein may be realized in digital electronic circuitry, integrated circuitry, specially designed application specific integrated circuits (ASICs), computer hardware, firmware, software, and/or combinations thereof. These various implementations may include implementation in one or more computer programs that may be executable and/or interpretable on a programmable system including at least one programmable processor, which may be special or general purpose, coupled to receive data and instructions from, and to transmit data and instructions to, a storage system, and at least one peripheral device.

These computer programs, which may also be referred to as programs, software, applications, software applications, components, or code, may include machine instructions for a programmable processor, and may be implemented in a high-level procedural and/or object-oriented programming language, and/or in assembly machine language. As used herein, the term “non-transitory computer-readable medium” refers to any computer program, product, apparatus, and/or device, such as magnetic discs, optical disks, memory, and Programmable Logic Devices (PLDs), used to provide machine instructions and/or data to a programmable processor, including a non-transitory computer-readable medium that receives machine instructions as a computer-readable signal. The term “computer-readable signal” refers to any signal used to provide machine instructions and/or data to a programmable processor. To provide for interaction with a user, the subject matter described herein may be implemented on a computer having a display device, such as a cathode ray tube (CRD), liquid crystal display (LCD), light emitting display (LED) monitor for displaying information to the user and a keyboard and a pointing device, such as a mouse or a trackball, by which the user may provide input to the computer. Displays may include, but are not limited to, visual, auditory, cutaneous, kinesthetic, olfactory, and gustatory displays, or any combination thereof.

Other kinds of devices may be used to facilitate interaction with a user as well. For instance, feedback provided to the user may be any form of sensory feedback, such as visual feedback, auditory feedback, or tactile feedback; and input from the user may be received in any form including, but not limited to, acoustic, speech, or tactile input. The subject matter described herein may be implemented in a computing system that includes a back-end component, such as a data server, or that includes a middleware component, such as an application server, or that includes a front-end component, such as a client computer having a graphical user interface or a Web browser through which a user may interact with the system described herein, or any combination of such back-end, middleware, or front-end components. The components of the system may be interconnected by any form or medium of digital data communication, such as a communication network. Examples of communication networks may include, but are not limited to, a local area network (“LAN”), a wide area network (“WAN”), metropolitan area networks (“MAN”), and the internet.

The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For instance, the implementations described above can be directed to various combinations and subcombinations of the disclosed features and/or combinations and subcombinations of several further features disclosed above. In addition, the logic flow depicted in the accompanying figures and/or described herein do not necessarily require the particular order shown, or sequential order, to achieve desirable results. It will be readily understood to those skilled in the art that various other changes in the details, devices, and arrangements of the parts and method stages which have been described and illustrated in order to explain the nature of this inventive subject matter can be made without departing from the principles and scope of the inventive subject matter.

What is claimed is:

1. A system for facilitating virtual medical checkups comprising:
a medical fueling/charging station having a display,
wherein a control board of said display is configured to receive first image data and second image data,
wherein a display user interface having a plurality of display windows is configured to present said first image data and said second image data within said plurality of display windows via said display,
a first computing device having a first user interface and operably connected to said control board of said display,
wherein said first computing device is configured to transmit said first image data of said first user interface to said control board,
wherein said first image data contains at least one of user data and patient data of a user profile,
a second computing device having a second user interface and operably connected to said control board of said display,
wherein said second computing device is configured to transmit said second image data of said second user interface to said control board,
wherein said second image data contains at least one of said user data and said patient data of said user profile,
a medical device operably connected to said control board,
wherein said medical device collects patient data from a user,
a processor operably connected to said medical fueling/charging station, first computing device, second computing device, and medical device, and
a non-transitory computer-readable medium coupled to said processor,
wherein said non-transitory computer-readable medium contains instructions stored thereon, which, when executed by said processor, cause said processor to perform operations comprising:
connecting said first computing device to said medical fueling/charging station,
receiving a medical checkup request from said first computing device,
connecting said second computing device to said medical fueling/charging station,
receiving said patient data from said medical device, and
presenting said first image data and said second image data on said display.

2. The system of claim 1, further comprising a secondary security device operably connected to said control board,
wherein said secondary security device is configured to communicate with said control board of said display.

3. The system of claim 2, wherein said secondary security device is configured to collect biometric data from a user, wherein said biometric data is used by said control board to verify login credentials of said user, wherein said login credentials allow said user to access said display user interface of said display.

4. The system of claim 3, further comprising additional instructions stored on said non-transitory computer-readable medium, which, when executed by said processor, cause said processor to perform additional operations comprising:
receiving said biometric data from said secondary security device,
checking said biometric data against said login credentials, and
activating said display when said biometric data matches said login credentials.

5. The system of claim 2, wherein said secondary security device is configured to send a computer readable signal to said control board, wherein said computer readable signal is used by said control board to verify login credentials of a user, wherein said login credentials allow said user to access said display user interface of said display.

6. The system of claim 5, further comprising additional instructions stored on said non-transitory computer-readable medium, which, when executed by said processor, cause said processor to perform additional operations comprising:
receiving said computer readable signal from said secondary security device,
checking said computer readable signal against said login credentials, and
activating said display when said computer readable signal matches said login credentials.

7. The system of claim 1, further comprising an input device,
wherein said input device is at least one of a camera or a microphone connected to at least one of said first computing device or second computing device,
wherein said camera is configured to collect video data of at least one of a healthcare provider and a patient,
wherein said microphone is configured to collect audio data of at least one of said healthcare provider and said patient.

8. The system of claim 7, further comprising additional instructions stored on said non-transitory computer-readable medium, which, when executed by said processor, cause said processor to perform additional operations comprising:
receiving at least one of said video data or said audio data from said input device, and
analyzing, using a machine learning technique, said video data or said audio data for at least one of user data or patient data.

9. The system of claim 1, further comprising additional instructions stored on said non-transitory computer-readable medium, which, when executed by said processor, cause said processor to perform additional operations comprising:
analyzing, using a machine learning technique, said user data and said patient data to determine a medical condition of a user.

10. The system of claim 9, further comprising additional instructions stored on said non-transitory computer-readable medium, which, when executed by said processor, cause said processor to perform additional operations comprising:
presenting said medical condition of said user on said second computing device.

11. A system for facilitating virtual medical checkups comprising:
a medical fueling/charging station having a first display,
wherein a first control board of said first display is configured to receive first image data and second image data,
wherein a first display user interface having a first plurality of display windows is configured to present said first image data and said second image data within said first plurality of display windows via said first display,
a second display having a second control board and operably connected to said first display,
wherein said second control board of said second display is configured to receive said first image data and said second image data,
wherein a second display user interface having a second plurality of display windows is configured to present said first image data and said second image data within said second plurality of display windows via said second display,
a first computing device having a first user interface and operably connected to said first control board of said first display,
wherein said first computing device is configured to transmit said first image data of said first user interface to said first control board,
wherein said first image data contains at least one of user data and patient data of a user profile,
a second computing device having a second user interface and operably connected to said second control board of said second display,
wherein said second computing device is configured to transmit said second image data of said second user interface to said second control board,
wherein said second image data contains at least one of said user data and said patient data of said user profile,
a medical device operably connected to said first control board,
wherein said medical device collects patient data from a user,
a processor operably connected to said first display, second display, first computing device, second computing device, and medical device, and
a non-transitory computer-readable medium coupled to said processor,
wherein said non-transitory computer-readable medium contains instructions stored thereon, which, when executed by said processor, cause said processor to perform operations comprising:
connecting said first computing device to said first display,
connecting said second computing device to said second display,
connecting said first display and said second display,
receiving first image data from said first computing device,
receiving second image data from said second computing device,
transmitting said first image data to said second display from said first display,
transmitting said second image data to said first display from said second display,
presenting said first image data and said second image data within a first plurality of display windows of said first display, and
presenting said first image data and said second image data within a second plurality of display windows of said second display.

12. The system of claim 11, further comprising a secondary security device operably connected to said first control board,
wherein said secondary security device is configured to collect biometric data from said user,
wherein said biometric data is used by said first control board to verify login credentials of said user,
wherein said login credentials allow said user to access said first display user interface of said first display.

13. The system of claim 12, further comprising additional instructions stored on said non-transitory computer-readable medium, which, when executed by said processor, cause said processor to perform additional operations comprising:
receiving said biometric data from said secondary security device,
checking said biometric data against said login credentials, and
activating said first display when said biometric data matches said login credentials.

14. The system of claim 13, wherein said secondary security device is configured to send a computer readable signal to said first control board, wherein said computer readable signal is used by said first control board to verify login credentials of a user, wherein said login credentials allow said user to access said first display user interface of said first display.

15. The system of claim 14, further comprising additional instructions stored on said non-transitory computer-readable medium, which, when executed by said processor, cause said processor to perform additional operations comprising:
receiving said computer readable signal from said secondary security device,
checking said computer readable signal against said login credentials, and
activating said first display when said computer readable signal matches said login credentials.

16. The system of claim 11, further comprising an input device,
wherein said input device is at least one of a camera or a microphone connected to at least one of said first display or second display,
wherein said camera is configured to collect video data of at least one of a healthcare provider or a patient,
wherein said microphone is configured to collect audio data of at least one of said healthcare provider or said patient.

17. The system of claim 16, further comprising additional instructions stored on said non-transitory computer-readable medium, which, when executed by said processor, cause said processor to perform additional operations comprising:
receiving at least one of said video data or said audio data from said input device, and
analyzing, using a machine learning technique, said video data or said audio data for at least one of user data or patient data.

18. The system of claim 11, further comprising additional instructions stored on said non-transitory computer-readable medium, which, when executed by said processor, cause said processor to perform additional operations comprising:
analyzing, using a machine learning technique, said user data and said patient data to determine a medical condition of said user.

19. The system of claim 18, further comprising additional instructions stored on said non-transitory computer-readable medium, which, when executed by said processor, cause said processor to perform additional operations comprising:
presenting said medical condition of said user within said second plurality of display windows of said second display.

20. A method for facilitating virtual medical checkups comprising:
connecting a first computing device to a first control board of a first display that is incorporated into a medical fueling/charging station,
wherein a first display user interface of said first display is configured to present first image data and second image data within a first plurality of display windows,
wherein said first image data contains user data and patient data of a user profile,
connecting a second computing device to a second control board of a second display,
wherein a first display user interface of said first display is configured to present first image data and second image data within a first plurality of display windows,
wherein said second image data contains said user data and said patient data of said user profile,
receiving medical data from a medical device operably connected to said first control board,
transmitting said first image data from said first computing device to said first display,
transmitting said second image data from said second computing device to said second display,
transmitting said first image data to said second display from said first display,
transmitting said second image data to said first display from said second display, and
analyzing, using a machine learning technique, said user data and said patient data of said first image data, second image data, and said medical device to determine a medical condition of a user of said first computing device and said medical device.
